(19) United States
(12) Patent Application Publication
(10) Pub. No.: US 2022/0319669 A1
(43) Pub. Date: Oct. 6, 2022

(54) GUIDING A THERAPEUTIC PSYCHEDELIC EXPERIENCE USING EXTENDED REALITY

(71) Applicant: RESURGENT PHARMACEUTICALS, INC., Minneapolis, MN (US)

(72) Inventor: Eric Greenbaum, Minneapolis, MN (US)

(21) Appl. No.: 17/220,738

(22) Filed: Apr. 1, 2021

(51) Int. Cl. G16H 20/70

(52) U.S. Cl. CPC G16H 20/70

(57) ABSTRACT

Embodiments of a method and system for XR-assisted therapeutic psychedelic experience include receiving a set of inputs from a participant at an interaction engine associated with an XR system; at the interaction engine, determining a participant model for the participant; at the interaction engine, determining therapeutic goals for the participant; at the interaction engine, generating an interaction plan including a conversation component and an augmented or immersive element component, based on the participant model and the therapeutic goals; and executing the interaction plan with the XR system, thereby promoting a therapeutic psychedelic experience, such as a mystical-type experience, associated with improved outcomes for the mental or emotional well-being of the participant.

### BACKGROUND

The US Food and Drug Administration (FDA) estimates the unmet need for treatment of mental health and mood disorders is extremely large, with potentially billions of people requiring therapeutic interventions. The use of psychedelics for healing or spiritual purposes is not new. Developing psychedelic therapy to meet the present need presents several logistical issues, however. Standardizing the psychedelic experience to improve mental wellness of a large and diverse population is a daunting challenge. While the development of formulations with precise and predictable onsets is ramping up, the limited availability of therapists to guide participants during the experience creates a bottleneck for wider use of psychedelics as medicine. During a psychedelic experience, a participant may experience significant fear and/or transient ideas of paranoia. Under unmonitored conditions, these effects may escalate to panic and dangerous behavior. The clinical setting offers some advantages for treating multiple subjects concurrently, but risks suboptimal experiences due to the interplay between the clinical environment, the subject's internal state, and the substances. New approaches are needed to make monitored psychedelic experiences and psychedelic-enhanced therapy more widely available.

### SUMMARY

The present disclosure describes systems and methods for an Extended Reality (XR)-assisted therapeutic psychedelic experience. The systems and method provided are configured to promote a therapeutic psychedelic experience, such as a mystical-type experience, associated with improved outcomes for the mental or emotional well-being of the participant, and thereby facilitate oversight of a therapeutic psychedelic experience. An XR-guided therapeutic psychedelic experience implemented via the methods and systems described herein can relieve the logistical strain and increase access to psychedelic-enhanced therapy.

In one aspect, embodiments of the present disclosure include a system for an Extended Reality (XR)-guided therapeutic psychedelic experience comprising: an XR system operable to execute a first interaction model for communicating with a first participant of a psychedelic experience, the XR system comprising: an input device operable to receive inputs from the participant; an output device operable to present a communication to the participant based on a first conversation component of the first interaction model; and an actuatable element operable to present an augmented or immersive element based on an augmented or immersive element component of the first interaction model; and an interaction engine operable to: determine a participant-XR system interaction model associated with engagement between the participant and the XR system, based on the participant inputs; determine a first therapeutic goal associated with the participant, based on participant inputs; and generate the first interaction model comprising the first conversation component and the augmented or immersive element component based on the participant-XR system interaction model and the first therapeutic goal. The input device can include a biofeedback sensor. The biofeedback sensor can be selected from the group consisting of brain activity sensors, muscle activity sensors, skin temperature sensors, heart rate sensors, respiratory rate sensors, eye movement sensors, bioimpedance sensors, and galvanic response sensors, or a combination thereof. The system can be further operable to execute a primary guide interaction model for communicating with a primary guide supporting the participant based on biofeedback, wherein the output device is further operable to present communication to the participant from the primary guide and the actuatable element is further operable to present augmented or immersive elements based on an augmented or immersive element component of the primary guide interaction model. The interaction engine can be further operable to determine a participant model comprising at least one of the following: personality traits of the participant, emotional states of the participant, biographical information of the participant, and a medical history of the participant; and wherein the first interaction model is further based on the participant model. The input device can be operable to receive at least one of the following inputs from or about the participant: biometric data, biographical information, relationship information, demographic information, preferences and dislikes for digital elements of the augmented or immersive component elements, preferences and dislikes for conversational components, musical tastes, religious or spiritual affiliation, and location. The interaction engine can be operable to: generate the first interaction model using an interaction tree comprising: an object associated with a first set of conversation components and a first set of augmented or immersive element components for presenting content; and a set of sub-objects connected to the object, each connection associated with a different participant response to the content; and select the first conversation component from the first set of conversation components and the first augmented or immersive element component from the first set of augmented or immersive element components based on the participant-XR system interaction model. The system can further include at least one additional XR system to execute at least one additional interaction model for interacting with a second participant of a second psychedelic experience, or a plurality of psychedelic experience participants, the at least one additional interaction model comprising at least one additional conversation component and at least one additional augmented or immersive element component tailored to the second participant or each of the plurality of participants based on a at least one additional participant-XR system interaction model and at least one additional therapeutic goal. The at least one additional XR system further comprises a biofeedback sensor. The system can be further operable to execute a primary guide interaction model for communicating with a primary guide supporting the second participant based on biofeedback, wherein the at least one additional XR system is further operable to present communication to the second participant or the plurality of participants from the primary guide and the actuatable element is further operable to present augmented or immersive elements based on an augmented or immersive element component of the primary guide interaction model.

In a second aspect, embodiments of the present disclosure include a method of improving a therapeutic outcome of a psychedelic experience comprising: receiving, at a remote interaction engine, inputs of a first participant of a psychedelic experience collected at a first XR system in response to outputting conversational audio for the first participant at a speaker of the first XR system; refining, at the remote interaction engine, a first participant-XR system interaction model based on the first participant inputs; refining, at the remote interaction engine, a first therapeutic goal based on the first participant inputs; generating a first interaction plan comprising a conversation component, based on the first participant-XR system interaction model and the first therapeutic goal; transmitting the first interaction plan from the remote interaction engine to the first XR system; and outputting, at the speaker of the first XR system, updated conversational audio based on the conversation component of the first interaction plan. The first participant inputs can include psychedelic agent data and biofeedback. The biofeedback can include at least one of the following parameters: speech pattern, brain activity, respiratory rate, heart rate, muscle activity, electrodermal, skin temperature, eye movement tracking, and motion detection. The step of refining the first participant-XR system interaction model can include: sensing a current value for a biofeedback parameter of the first participant and comparing the current value with stored biofeedback parameter limits of the first participant; and communicating the current value of the first participant biofeedback parameter to a primary guide through the XR system if the current value is outside the stored limits; or retransmitting the first interaction plan from the remote interaction engine to the XR system if the current value is within the stored limits. The method can further include generating an analysis of the efficacy of the first interaction plan for achieving the first participant therapeutic goal; generating, at the remote interaction engine, a second interaction plan for achieving a second participant psychotherapy goal associated with a second participant of a second psychedelic experience, based on the analysis; and executing the second interaction plan with a second XR system associated with the second participant. The step of generating the analysis can include receiving responses to a questionnaire completed by the first participant after the psychedelic experience. The method can further include receiving, at the remote interaction engine, second participant inputs collected at the second XR system in response to executing the second interaction plan for the second participant; determining a second participant-XR system interaction model based on the second participant inputs and the first participant-XR system interaction model; and updating the second interaction plan based on the second participant-XR system interaction model. The method can further include determining a participant model comprising a personality model associated with at least one of the group consisting of personality traits of the first participant, a mood model associated with emotional states of the first participant, a biographical model associated with contextual information of the first participant, and a medical model associated with a medical history of the first participant, and the first participant-XR system interaction model; and determining a therapeutic goal, based on the first participant inputs, wherein generating the first interaction plan is further based on the participant model. The method can further include receiving primary guide inputs associated with the psychedelic experience of the first participant; and determining a primary guide model based on the primary guide inputs, wherein generating the first interaction plan is further based on the primary guide model.

In a third aspect, embodiments of the present disclosure include a non-transitory computer-readable storage medium that stores instructions for an interaction engine of an Extended Reality (XR) system for guiding a therapeutic psychedelic experience that, when executed by a processor, cause the interaction engine to: receive inputs from a participant of the therapeutic psychedelic experience; determine a participant-XR system interaction model associated with engagement between the participant and the XR system based on participant inputs; determine a first therapeutic goal associated with the participant based on participant inputs; and generate a first interaction model comprising a first conversation component and an augmented or immersive element component based on the participant-XR system interaction model and the first therapeutic goal.

Another aspect of the present disclosure includes a method of improving the therapeutic outcome of psychotherapy of a participant in need thereof, the method comprising: providing the participant with an XR system operable to execute a first interaction model for communicating with the participant, the XR system comprising: an input device operable to receive inputs from the participant; an output device operable to present a communication to the participant based on a first conversation component of the first interaction model; and an actuatable element operable to present an augmented or immersive element based on an augmented or immersive element component of the first interaction model; and an interaction engine operable to: determine a participant-XR system interaction model associated with engagement between the participant and the XR system, based on the participant inputs; determine a first psychotherapy goal associated with the participant, based on participant inputs; and generate the first interaction model comprising the first conversation component and the augmented or immersive element component based on the participant-XR system interaction model and the first psychotherapy goal; and administering a psychedelic agent (or instructing the participant to take the psychedelic agent); whereby the first interaction model improves the likelihood of evoking a mystical-type experience in the participant compared to the likelihood without the generation of a first interaction model. The first interaction model can be generated using a stored participant model that includes at least one of the group consisting of personality traits of the participant, contextual information of the participant, and a medical history of the participant. The psychedelic agent can be selected from the group consisting of 5-HT_{2A} agonists, empathogenic agents, dissociative agents, and combinations thereof. The psychedelic agent can include psilocybin, LSD, DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2′S,4′S)-(+)-9,10-Didehydro-6-methylergoline-8β-(trans-2,4-dimethylazetidide); 2C-BCB (4-Bromo-3,6-dimethoxybenzocyclobuten-1-yl) methylamine; ayahuasca; 3,4,5-trimethoxyphenethylamine (mescaline); 5-methoxy-N,N-dimethyltryptamine (5-meo-DMT); ibogaine; 3,4-methylenedioxymethamphetamine (MDMA); or ketamine. The psychedelic agent can be an extract containing one or more components of mescaline-containing cacti, psilocybin-containing mushrooms, cannabis, and DMT-containing chacruna leaves.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 is a block diagram depicting a method 100, for preparing a participant profile for implementing an Extended Reality (XR)-assisted therapeutic psychedelic experience, according to one or more embodiments of the present disclosure.

FIG. 2 is block diagram depicting a method 200, for establishing set and setting for a therapeutic psychedelic experience based on a prepared participant profile, according to one or more embodiments of the present disclosure.

FIG. 3 is block diagram depicting a method 300 for implementing XR-assisted therapeutic psychedelic experiences, at scale, according to one or more embodiments of the present disclosure.

### DETAILED DESCRIPTION

Embodiments of the present disclosure include methods and systems for guiding a therapeutic psychedelic experience using Extended Reality (XR). For example, a method of the present disclosure can include receiving a set of inputs from a participant at an interaction engine associated with an XR system; at the interaction engine, determining a participant model for the participant; at the interaction engine, determining therapeutic goals for the participant; at the interaction engine, generating an interaction plan including a conversation component and an augmented or immersive element component, based on the participant model and the therapeutic goals; and executing the interaction plan with the XR system, thereby promoting a therapeutic psychedelic experience, such as a mystical-type experience, associated with improved outcomes for the mental or emotional well-being of the participant.

### Definitions

The terms recited below have been defined as described below. All other terms and phrases in this disclosure shall be construed according to their ordinary meaning as understood by one of skill in the art.

As used herein, “therapeutic psychedelic experience” refers to improved mental or physical well-being induced by the action of a psychedelic agent. A therapeutic psychedelic experience can be initiated to by the individual taking a psychedelic agent (i.e., participant) for any reason. For example, a participant may seek to induce a therapeutic psychedelic experience to improve psychological well-being, improve a general emotional state, and/or reduce stress. In some cases, a participant seeks to induce a therapeutic psychedelic experience as part of treatment plan for an affective disorder, mood disorder, anxiety disorder, post-traumatic stress disorder, obsessive-compulsive disorder, substance abuse problem, drug addiction, and or addictive behavior, including relapse prevention thereof.

The methods and systems of the present disclosure use “Extended Reality (XR)”, which refers to various technologies capable of enhancing senses, whether by providing additional information about the actual world or creating a simulated world. XR includes Virtual Reality (VR), Augmented Reality (AR) and Mixed Reality (MR) technologies.

“Virtual reality (VR)” refers to a complete immersion experience.

“Augmented reality (AR)” refers to technology that adds digital elements to a live view.

“Mixed Reality (MR)” includes elements of both AR and VR. For example, in MR, real-world and digital objects can interact.

A “mystical-type experience” as used herein is an experience induced by the action of a psychedelic agent which is similar to a spontaneously occurring mystical experience. A mystical-type experience can have substantial and sustained personal meaning and spiritual significance marked by one or more of unity (either internal (transcendence of separation of oneself) and/or external (boundary dissolution between self and surroundings), transcendence of time and space, ineffability, sense of sacredness, noetic quality, and positive mood (joy). The intensity of the experience can be quantified using surveys and questionnaires described below.

The term “participant” as used herein refers to a patient or subject who has taken or plans to take one or more psychedelic agents (AKA “voyager”), by any suitable route for the specific psychedelic agent(s) and dose thereof.

“Integration” refers to a stage of a psychedelic experience that includes revisiting the experience itself (e.g., by discussion or re-immersion in one or more of the stimuli of the XR environment (e.g., music and/or scent), discussion of the meaning that the participant ascribes to the experience, and/or discussion of how the insights or realizations gained during the experience can be applied to daily life. The integration stage can further enhance a therapeutic outcome.

The term “guide” refers to the individual(s) or system component(s) overseeing or assisting with a psychedelic-assisted therapy session. Typically, active psychological support of a guide is not required while the participant is in an altered state induced by the psychedelic agent. A “primary guide” refers to an individual that has training and expertise overseeing psychedelic therapy, psychotherapy, and/or complex therapy. A primary guide can oversee the psychedelic therapy from a treatment facility associated with a hospital or research facility, a mental health clinic, or a retreat center. A primary guide can also provide or assist with screening, evaluation, and follow-up services. Competencies of a primary guide can include empathetic abiding presence; trust enhancement; spiritual intelligence; knowledge of the physical and psychological effects of psychedelics; therapist self-awareness and ethical integrity; and proficiency in complementary techniques.

An “automated guide” refers to a component of the XR system (e.g., a chatbot or other AI) configured to assist/support a primary guide's oversight of one or more participants by interacting with each participant via directed dialog or natural language processing and to alert the primary guide under preset conditions, and to develop and maintain rapport and trust to minimize the risk of adverse reactions to the psychedelic agent. Through use of automated guides, the methods and systems of the present disclosure are not inherently limited by primary guide time or attention, and may diminish the societal burden of treating mental health. An automated guide can have a long-term relationship with a participant, while assisting multiple primary guides who may have a short term relationship with a participant (e.g., a single session or part of a session). In some cases, a plurality of automated guides, of which each automated guide is in direct communication with a single participant, communicates with a primary guide thereby allowing a single primary guide to support multiple participants, concurrently. For example, a single primary guide may indirectly support 2, 3, 4, 5, 10, 15, or more participants during at least part of a psychedelic experience via use of automated guides in direct communication with each participant. The automated guide can have the same virtual presence (e.g., avatar, voice, speech patterns, etc.) as the primary guide, such that the participant cannot distinguish whether the primary guide or the automated guide is interacting with the participant. In other cases, the primary and automated guides are distinguishable. For example, if a participant is more comfortable sharing personal or intimate details about the experience with an automated guide than with the primary guide, there can be a therapeutic benefit conferred by modeling the automated guide so that it is distinguishable from the primary guide.

“Guided group session” refers to a shared psychedelic experience format that allows a plurality of participants to access one or more primary and automated guides, and/or offer participants an opportunity to share experiences. In some cases, the plurality of participants in a guided group session are immersed in the same specialized virtual environment to offer an opportunity to build a bond related to being similar situations and/or facing mutual challenges. Additionally or alternatively, each participant can be immersed a personalized virtual environment for at least part of the session while being aware of the presence of other participants via an augmented or immersive element (e.g., a visual, auditory, or olfactory stimulus associated with other participants).

As used herein, a “sitter” can assist the participant and/or the primary guide by being physically present with a participant during the experience. In some cases, a sitter can be a medically trained individual.

“Set and setting” refers to the inner and outer environments in which a drug experience takes place; “set” refers to the mind-set and expectations the participant brings to the experience, which can be shaped by preparation, and “setting” is the outward circumstances in which the experience takes place, e.g., physical surroundings, but also the atmosphere of the space for the session itself.

A “psychedelic agent” includes natural and synthesized conscious-altering substances capable of inducing an altered state of consciousness, i.e., a marked deviation in the subjective experience or psychological functioning of a normal individual from his or her usual waking consciousness. Psychedelic agents include 5-HT_{2A} agonists, empathogenic agents, and dissociative agents. 5-HT_{2A} agonists include psilocybin, LSD, DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2′S,4′S)-(+)-9,10-Didehydro-6-methylergoline-8β-(trans-2,4-dimethylazetidide); 2C-BCB (4-Bromo-3,6-dimethoxybenzocyclobuten-1-yl) methylamine; ayahuasca; 3,4,5-trimethoxyphenethylamine (mescaline); 5-methoxy-N,N-dimethyltryptamine (5-meo-DMT); and ibogaine. Empathogenic agents include serotonin (5-HT) releasing agents such as 3,4-methylenedioxymethamphetamine (MDMA). Exemplary dissociative agents include N-Methyl-D-aspartate (NMDA) receptor agonists such as ketamine. A psychedelic can include components of mescaline-containing cacti (e.g., peyote cactus, Echinopsis pachanoi), psilocybin-containing mushrooms (e.g., Teonanacatl), cannabis and/or preparations containing dimethyltryptamine (e.g., ayahuasca, yop), which are available as a fresh product or can be taken in dried form, and DMT-containing chacruna leaves.

### Extended Reality Assisted Therapeutic Psychedelic Experience

Embodiments of the present disclosure include methods and systems to improve management of a therapeutic psychedelic experience and progression toward the therapeutic goals of a participant. The improvement can be achieved by providing enhanced interactions with an Extended Reality (XR) system before, during, and/or after a psychedelic experience. The interaction can be facilitated or maintained via an automated guide. The methods and systems promote the establishment and maintenance of a positive interaction between the participant and the XR system before and during the psychedelic experience. In particular, the method and/or system can receive and process information related to a participant's personality, interests, treatment-related challenges, and goals over time, in order to drive personalized conversations and other interactions with the participant that resonate with the participant's unique personality and circumstances, and thereby improve the psychedelic experience. This personalization can optimize set and setting,

The system can additionally or alternatively process data from interactions between the XR system, the automated guide and/or interactions between additional automated guides and additional participants, to further refine models that guide outputs of one or more XR systems in improving future interactions between the XR system and the participants. For example, the system can be configured to evaluate how selected a virtual, augmented, or mixed environment impacts therapeutic effects or identify individual factors predictive of response to varying the XR environment (or properties of the environment and/or how selected music impacts therapeutic effects or identify individual factors predictive of response to varying musical genres or musical features other than genre to individualize session selections.

In a specific application, the method is implemented using an XR system integrated into a platform (e.g., a cloud-based system) for collecting and sharing real-time data about the progress of each participant toward his/her respective therapy-related goals. The platform can additionally or alternatively improve outcomes at population-wide and individual scales, with targeted analyses of data collected from each participant.

The methods and systems of the present disclosure can provide several benefits over conventional approaches for improving the therapeutic psychedelic experience. First, embodiments of the disclosure can effectively engage a participant in adapting automated guide communications to each participant at different points in time. Additionally or alternatively, the technology can leverage state-of-the-art practices for generating psychologically effective interaction plans (e.g., for communicating with a participant through conversation and immersion (e.g., in an augmented or virtual environment) and/or delivering treatment aids to the participant in real-time. The scientific, clinical, and anthropological/spiritual literature on the use of psychedelics emphasizes the importance of “set” and “setting”, which are integral to gaining the greatest therapeutic benefit, maintaining ethical boundaries, and avoiding untoward effects. The methods and systems described facilitate ensuring a familiar, secure, comfortable, and safe physical, psychological, and social environment for the therapeutic psychedelic experience because the session can be implemented outside of a clinical setting (e.g., a participant's home).

Second, embodiments of the disclosure can enable continuous monitoring or management of one or a plurality of participants outside of a medical setting, thereby facilitating increased efficacy of treatment and delivery of the therapeutic psychedelic experience at scale. The XR system (e.g., via the automated guide) can interact with the participant before, during, or after a therapeutic psychedelic experience in response to specific conditions (e.g., signs of distress) in a remote setting to help improve outcomes through education, facilitation of relaxation, surrender, acceptance of a treatment regimen, and/or personalized engagement tools. For example, the automated guide can be prepared to respond to signs of fear that arise in participant with gentleness and presence, while remaining neutral and nonjudgmental.

Third, embodiments of the disclosure can provide predictive biofeedback for a therapeutic psychedelic experience. For example, the system can be configured to generate simulated therapeutic psychedelic experience that will permit a primary guide, automated guide, or other therapy-associated user to assess whether a participant is ready to take a psychedelic agent. Generation of the simulated therapeutic psychedelic experience can include configuring the system to measure biofeedback during a therapeutic psychedelic experience (e.g., speech patterns, heart rate, respiratory rate, electrical bioimpedance, galvanic skin response, blood pressure, eye movement, body temperature, electrocardiogram (ECG), electroencephalogram (EEG), etc.). The system can collect biofeedback from at least one participant and correlate biofeedback with the desirability of the experience (e.g., based on responses to a questionnaire or clinician reports). The therapeutic-correlated biofeedback can be used as a reference for comparison with biofeedback collected during a participant's preparation stage of the experience, and provide a metric for determining whether set and setting for a therapeutic psychedelic experience has been established. The reference can be specific to a single participant or based on a population or subpopulation of participants (e.g., generalized from all participants, only healthy participants, or only participants seeking therapy for a specific affective disorder).

Fourth, embodiments of the present disclosure can leverage a network of XR systems and/or automated guides to collect data for generating insights into participants, drawing commonalities between participant populations, and/or other suitable purposes in improving therapeutic management (e.g., by a care provider). Based upon these insights, the XR system can be configured to detect patterns in one or more biofeedback parameters (e.g., speech patterns, heart rate, respiratory rate, electrical bioimpedance, galvanic skin response, blood pressure, eye movement, body temperature, electrocardiogram (ECG), electroencephalogram (EEG), etc.) in a participant's responses to the automated guide that are associated with an increased risk of a non-therapeutic psychedelic experience, and alert the primary guide (or other therapy associated user) to cancel or postpone the experience, thereby improving the likelihood of a therapeutic effect on a participant's psychological or emotional well-being.

Fifth, embodiments of the present disclosure can provide improvements to the functioning of computer-related technology. For example, one or more embodiments provide a distribution of functionality across a network of one or more XR systems (e.g., executing personalized interaction plans for communicating with and interacting with participants, based on historic conversations with participants, etc.), interaction engines (e.g., developing participant models and goals based on user inputs across users in order to use in generating personalized interaction plans), and/or any other components. In addition, methods of the present disclosure can improve computational accuracy of selecting conversation components and/or immersion components suited to achieving participant goals (e.g., based on analyzing efficacy of previously selected conversation components and/or immersion components in achieving the goals). The improved accuracy can lighten the computational processing load by enabling the automated guides to output fewer communications and/or immersion to achieve a given participant goal. However, the technology can provide any other suitable benefits in the context of using non-generalized systems to improve participant with the XR system.

A method for psychedelic-enhanced therapy includes utilizing a XR system to facilitate, initiate, maintain and/or enhance a therapeutic psychedelic experience, and thereby increase the likelihood that the participant achieves a mystical-type experience after taking a psychedelic agent. Setting design utilizing a XR system can provide the framework and basis for a wholesome, mind-altered state and reduce the risk of non-therapeutic experiences during the mind-altered state. The participant has little control over the course of the experience as the psychedelic takes effect, however, prior to taking the psychedelic agent, the participant's mental state can be assessed and improved. The method and XR system of the present disclosure can facilitate a mental state that is conducive to a therapeutic psychedelic experience as the participant prepares to take the psychedelic agent. The method and virtual reality system can facilitate a therapeutic integration of the experience to increase the potential for personal transformation.

Methods of the present disclosure can include receiving a set of user inputs from the participant at an interaction engine (e.g., computing system) associated with a XR system via an input device (e.g., keyboard, keypad, mouse, touchscreen, touchpad, joystick, remote control, microphone, camera, etc.). The inputs can be provided by the participant and/or a therapy-associated user (e.g., healthcare entity, therapist, spiritual advisor, or sitter). The inputs can be received through sensors and/or input devices integrated into a XR system. For example, inputs can be received from optical sensors (e.g., of a camera module, of a video module) of the XR system, audio sensors (e.g., of a microphone unit) of the XR system, and a user interface (e.g., a touch screen/touch pad for engaging the user in text-based conversations) integrated in the XR system. In some cases, inputs are received from a supplementary device (e.g., a wearable device) associated with the participant (e.g., directly using a wireless data link, indirectly using a wireless data link, through application programming interfaces, through health data aggregation applications of a mobile device, etc.). Receiving can include querying one or more supplementary devices (e.g., through wireless communication, API requests tailored to different supplementary devices, etc.) with data requests for data associated with the participant. In some cases, the inputs include electronic health records (EHRs) of the participant.

Received inputs can include inputs related to the personality of the participant, the mood/emotional state of the participant, the spiritual/religious affiliation, biographical information of the participant, medical information of the participant, and/or any other suitable inputs. The inputs can be directly provided by the participant, and/or can additionally or alternatively be indirectly provided by any other suitable means. Furthermore, inputs of the above listed categories can overlap across categories, can inform inputs of other categories, and/or can be derived from inputs of multiple categories in any suitable manner.

Inputs related to the personality of the participant can be informative of personality traits of the participant (e.g., openness, experience, conscientiousness, extraversion, and neuroticism within a five factor model, other factors from other personality trait models, etc.) and/or can be informative of the personality types (e.g., according to a Myers Briggs categorization, etc.) of the participant. Personality-related inputs may be associated with a likelihood of a therapeutic psychedelic experience. Inputs informative of the personality of the participant can include inputs from EHRs of the participant informative of personality traits and/or types; inputs received at a touch screen, audio sensors, and/or image sensors integrated with the XR system related to conversation content between the participant and the primary guide (or automated guide). In some cases, inputs are received at a smartphone and/or other personal device of the participant. However, personality-related inputs can be defined and/or derived in any manner.

Inputs related to the mood/emotional state of the participant can be informative of a temporary mood/emotional state of the participant, and can be derived from facial expressions of the participant captured by image sensors, speech (e.g., speech content, speech tone, etc.) captured by audio sensors, speech captured from inputs at a touch screen of the XR system, speech captured in any other suitable manner, analysis of events of the participant (e.g., from biographical data extracted from conversations with the participant, from biographical data extracted from posts associated with the participant in electronic social networking applications, from digital communication received and/or transmitted to personal devices of the participant, etc.), and/or any other suitable source. Mood/emotional state-related inputs may be associated with a likelihood of a therapeutic psychedelic experience.

Inputs related to biographical information of the participant can be contextual information of the participant (e.g., name, appearance, etc.), relationship information of the participant (e.g., family information, social network information, professional network information, relationship status, etc.), demographic information of the participant (e.g., nationality, ethnicity, age, gender, etc.), interests of the participant (e.g., likes, dislikes, hobbies, etc.), musical tastes of the participant (e.g., preferred genre of music for relaxation), religious or spiritual affiliation of the participant (e.g., frequency or regularity of prayer, attendance at religious or spiritual gatherings/ceremonies, life events of the participant (e.g., regular events, irregular events, etc.), locations of the participant (e.g., places relevant to the life of the participant), and any other suitable biographical information, biological information-related inputs may be associated with a likelihood of a therapeutic psychedelic experience User inputs related to biographical information can be obtained in any manner described above, and/or in any suitable manner.

Inputs related to medical information of the participant can be informative of: medication regimens of the participant, side effects of medications of the participant, interactions between medications of the participant, allergies of the participant, conditions of the participant, mental health of the participant, mobility of the participant, exercise behavior of the participant, diet of the participant, weight of the participant, medical history of the participant, other treatment regimens of the participant, preferred medical providers of the participant (e.g., hospitals, pharmacies, clinics, caretakers, etc.), medical device data (e.g., datasets collected with medical devices, historical medical device types that the participant has used, current medical device types, etc.), and/or any other suitable medical information. User inputs related to medical information can be extracted in any suitable manner. For example, a medical history of epilepsy, bipolar disorder, schizophrenia, or other psychotic disorders, first-degree relatives (as parent or full sibling) with past or present psychiatric disorders, including schizophrenia, bipolar affective disorder and other psychoses, requiring concomitant treatment with anti-psychotic medications, prescribed for the management of either psychiatric symptoms or nausea, uncontrolled hypertension, baseline laboratory values indicative of severely compromised hepatic function, women who are pregnant or nursing, or of child bearing potential and are not practicing an effective means of birth control, a serious suicide or homicide risk, risk for psychiatric hospitalization, inability to fully understand the potential risks and benefits of the therapy and give informed consent can indicate a participant is unsuited for psychedelic-assisted psychotherapy during the inquiry stage.

A participant can be characterized in the system by one or more of their medical condition, demographic information (e.g., gender, age, marital status, ethnicity, nationality, socioeconomic status, sexual orientation, etc.), living situations (e.g., living alone, living with pets, living with a significant other, living with children, etc.), dietary habits (e.g., omnivorous, vegetarian, vegan, sugar consumption, acid consumption, etc.), behavioral tendencies (e.g., levels of physical activity, drug use, alcohol use, etc.), level of mobility (e.g., related to distance traveled within a given time period), history psychedelic use and/or any other suitable trait that is relevant to psychedelic therapy. In one specific example, the participant can be characterized as a member of a population of participants having a specific condition (e.g., elderly participants, cancer patient participants, and participants diagnosed with a depressive disorder, anxiety disorder, obsessive compulsive disorder, etc.). Therapeutic models and interaction plans can be determined and/or executed in different manners for different populations.

Receiving user inputs can be performed at predetermined time intervals (e.g., retrieving and transmitting user input data at specific stages of a therapeutic psychedelic experience), in response to and/or concurrently with a specific condition (e.g., a new set of user inputs from a participant interacting with the automated guide or XR system, a threshold amount and/or types of user inputs). Thus, receiving user inputs can include scheduling data requests for user input data. For example, inputs can be received at an inquiry stage, a preparation stage, a psychedelic experience stage, and/or an integration stage.

An inquiry stage can include receiving inputs that facilitate determining whether a participant is a candidate for psychedelic-assisted psychotherapy (e.g., standard personality tests and medical history). In some cases, the inquiry stage can include immersing the participant in a simulated psychedelic XR environment, biofeedback can be collected and compared with biofeedback results of individuals known to be good candidates for therapeutic psychedelic experiences. The collected data can be utilized to refine XR-system engagement models to expand access.

A preparation stage can include receiving inputs to establish baselines and thresholds for one or more physiological parameters. The preparation stage can include receiving inputs that facilitate assessing the mindset of the participant before taking a psychedelic agent (e.g., responses to questions and/or biofeedback indicative of a relaxed or agitated mental state such as speech patterns, heart rate, respiratory rate, electrical bioimpedance, galvanic skin response, blood pressure, eye movement, body temperature, electrocardiogram (ECG), electroencephalogram (EEG), etc.) and determine whether to proceed with, postpone or cancel a scheduled psychedelic session based on the input. Preparation can include assessing the physiological response to a simulated psychedelic experience, where certain physiological parameters would indicate readiness to take the psychedelic agent. Inputs received in the preparation stage can also facilitate estimating the duration and intensity of the psychedelic experience (e.g., inputs related to the type and dose of psychedelic). The preparation stage can span hours, days, or weeks. For example, this stage can involve one or more sessions of receiving inputs for generating and refining a participant model to optimize set and setting for the psychedelic experience. Biofeedback can also be used directly as an output to prompt the participant to change physiological reactions by controlling thoughts, emotions or behavior (e.g., taking deep breaths).

The psychedelic experience stage can include receiving inputs (e.g., one or more of the biofeedback parameters described above) after taking the psychedelic agent during the experience induced by the psychedelic agent. Inputs can be received at a pre-determined frequency to monitor the participant's response to the psychedelic agent (e.g., every 30 minutes for about 6-10 hours). The inputs can be received via any suitable sensor during this stage. The sensor can be the same or different than a sensor used during the preparation stage. For example, during preparation inputs can be received from a sensor integrated in a head mounted display (HMD), while during the psychedelic experience inputs can be received from interactive computer programs, earbuds, headbands, wearable haptics, non-wearable haptics (e.g., haptic joystick), mobile smart device (e.g., web camera, speaker with microphone, and/or phone), or other sensor present near the participant permitting the participant to remove the HMD during the experience (e.g., during the peak psychedelic experience).

The integration stage can include receiving inputs at the end of the psychedelic experience that facilitate refinements to one or more of the participant's profiles (e.g., responses used to identify a subjective experience such as the “Mystical Experience Questionnaire” (MEQ30), Beck Depression Inventory, “Questionnaire for recording extraordinary states of consciousness' (5D-ABZ), “Altered States of Consciousness Rating Scale”, “Phenomenology of Consciousness Inventory”, “Hallucinogen Rating Scale” (HRS 3.06), “Addiction Research Inventory”, “States of Consciousness Questionnaire”, “Persisting Effects Questionnaire”, “Psychotomimetic States Inventory”, “Ego Dissolution Inventory (EDI)”, “Hood Mysticism Scale”, “Mystical Experience Questionnaire (MEQ43)”, “States of Consciousness Questionnaire” (SOCQ), “Persisting Effects Questionnaire”). The responses to the diagnostic surveys which may facilitate discussions during subsequent psychotherapy sessions with a therapy-associated user (e.g., the therapist that recommended psychedelic-assisted therapy). In one or more embodiments, the participant experiences or reports experiencing freedom from the limitations of the participant's personal self and feeling a unity or bond with what was felt to be greater than the participant's personal self, pure being and pure awareness (i.e., beyond the world of sense impressions), oneness in relation to an “inner world” within, the fusion of your personal self into a larger whole, unity with ultimate reality, eternity or infinity, oneness or unity with objects and/or persons perceived in your surroundings, insight that “all is One”, awareness of the life or living presence in all things, gain of insightful knowledge experienced at an intuitive level, certainty of encounter with ultimate reality (in the sense of being able to “know” and “see” what is really real at some point during the experience, certainty now or during the experience that the ultimate reality was encountered (i.e., that the participant “knew” and “saw” what was really real), a sense of being at a spiritual height, a sense of reverence, and/or feeling that the participant experienced something profoundly sacred and holy. In one or more embodiments, the participant experiences or reports experiencing amazement, feelings of tenderness and gentleness, feelings of peace and tranquility, an experience of ecstasy, a sense of awe or awesomeness, and feelings of joy. In one or more embodiments, the participant experiences or reports experiencing transcendence of time and space. For example, the participant may indicate loss of the usual sense of time, loss of the usual sense of space, loss of usual awareness of where the participant was, a sense of being “outside of time, beyond past and future, a sense of being in a realm with no space boundaries, and/or an experience of timelessness. In one or more embodiments, the participant experiences or reports experiencing ineffability. For example, the participant can indicate an inability to describe the experience in words, or that the experience cannot be described adequately in words, the feeling that describing the experience in words does not “do it justice”, a feeling that it would be difficult to communicate the experience to others who have not had similar experiences.

In a specific example, user inputs received in real-time can be used for updating an interaction plan, controlling supplementary devices, and initiating telecommunication. Receiving user inputs can include applying natural language processing algorithms to extract a sentiment from conversational user inputs collected at a microphone of the XR system; determining that the sentiment satisfies an emergency situation condition (e.g., the participant is increasingly distressed); and transmitting the user conversational inputs (and/or other associated data) in response to detecting the emergency situation condition. The interaction plan can be configured to identify the risk to the participant from the detected emergency situation and communicate with the appropriate personnel or system component. The interaction plan can include at least one on-call medical professional that can respond in-person as required based on the detected emergency situation. The number of medical professionals can be varied based on the number of participants under the influence of a psychedelic agent and/or scheduled to be under the influence of a psychedelic agent (e.g., one medically trained sitter on-call for every 5, 10, 15 or more, participants). The interaction plan can be configured to detect an immediately life-threatening emergency situation condition and respond by contacting emergency response personnel (e.g., EMT). Inputs in one category of the participant model (e.g., personality model category, mood category, biographical model category, medical model category, interaction stage model category) can be derived from other inputs in the same category and/or inputs from other categories of the participant model. Inputs can overlap with or otherwise inform inputs of other categories of the participant model.

Methods of the present disclosure can include determining one or more participant models for the psychedelic experience at the interaction engine. The method can include determining and/or refining models that cooperatively govern output of the automated guide and/or one or more components of the XR system for engaging the participant. For example, participant goals (e.g., intention for the session, achieving a mystical-type experience) and interaction plans can be used to ultimately improve outputs of the XR system, in promoting the therapeutic psychedelic experience, including improving the likelihood of a mystical-type experience.

The participant model can include a personality model category, a mood category, a biographical model category, a medical model category, and an interaction stage model category (e.g., a participant-XR system interaction model) related to extent of engagement between the participant and the XR system (e.g., the virtual environment). Refining the participant model can include iteratively refining hypotheses of factors of each category of the participant model based on the inputs above (e.g., the personality model category can have factors associated with personality traits, personality types of the participant, and/or other personality-related factors; the mood category can have factors associated with transient emotional states of the participant and/or other mood-related factors; etc.). The interaction stage model category can have factors associated with duration of time of the interaction between the automated guide and the participant, frequency, openness, and regularity of interactions between the XR system and the participant, willingness of the participant to allow an XR system to aid in achieving participant goals, willingness of the participant to allow an XR system to communicate with therapy-associated users (e.g., care providers or sitter) about the participant, and/or other interaction-related factors.

Determining and refining the personality model category of the participation model can include processing new direct and indirect inputs associated with traits and types of the participant, and updating the current hypothesized personality model of the participant, and whether or not the participant is engaged with the virtual environment, or has surrendered to the psychedelic experience based on the new inputs. For example, a participant can be hypothesized to have a low amount of agitation based on a propensity to talk to the automated guide; however, this hypothesis can be refined in upon receiving a higher frequency of talking interactions.

Determining and refining the mood features of the participant model can include processing new direct and indirect inputs to extract facial expressions of the participant (e.g., from video data), speech (e.g., speech content, speech tone, etc.) of the participant (e.g., from inputs provided a touch pad integrated with the XR system, from inputs received at a microphone integrated with the system (e.g., headset), inputs associated with biographical data of the participant (described in further detail below), and any other suitable inputs. Features extracted from the inputs can include word choices selected by the participant, motions performed by the participant, amount of “small talk” that the participant is willing to engage in, average mood of the participant, personality traits of the participant, mood patterns of the participant, mood correlations of the participant (e.g., in relation to biographical events of the participant described below, etc.), mental health of the participant, and other factors associated with mood of the participant. In a specific example of refinement of a mood factor, the participant can be hypothesized to be nervous about initiating a therapeutic psychedelic experience, and this hypothesis can be validated and refined upon consistently observing facial expressions of the participant revealing the participant consistently has a pattern of nervousness, which in turn, can facilitate interactions with the XR system that promote relaxation (e.g., actuation of augmented or immersive elements such as the sounds (e.g. quiet, music, rhythm, humming, etc.), images (e.g., environments, colors, patterns, effects, etc.), smells (e.g. scent generation by a companion device or components embedded in a virtual or augmented reality headset system), light (e.g. control of dimmable or indirect light) based on a participant model and/or conversational elements with reassuring language from the automated guide).

Determining and refining the medical model for a participant can include processing new direct and indirect inputs associated with a participant's medication schedule, side effects of medications, condition, allergies, mobility, mental health, exercise behavior, diet, weight, and medical history, and inputs associated with one or more of the personality model, the mood, and the biographical model of the participant.

The method can include identifying correlations between indirect and direct inputs of the participant (across different factors of the personality model, the mood model, the biographical model, and the medical model), predicting the metal state of the participant, anticipating whether the participant will have a non-therapeutic response after taking the psychedelic agent, adapting a prior schedule of interactions between the automated guide, or components of the XR system (e.g., sensors) and the participant, switching between operation modes of reminding the participant that the automated guide is present vs. checking in with the participant (e.g., soliciting a response related to current state); identifying which participant types specific interactions of the automated guide and/or XR system have the most impact on the likelihood of a mystical-type experience, observing behavior change patterns, assessing readiness for behavior change, and any other suitable refinement.

Determining and refining a participant-XR interaction model can be based on interaction features including any one or more conversation features (e.g., frequency of conversations; duration of conversations, such as average duration; speed of participant response to communications by the automated guide; conversation content; conversation tone; participant mood over the course of the psychedelic experience, such as derived from facial expression analysis; comparisons between expected conversations and actual conversations; participant-initiated conversations vs. automated guide-initiated conversations; frequency of the XR system misunderstanding the participant), cross-participant features (e.g., engagement or surrender level relative to other participants; conversation content for one participant versus another participant; other suitable comparisons of interaction features between participant; etc.); goal-related features (e.g., willingness of participant receive support from an automated guide for achieving participant goals; number and/or types of goals achieved in cooperation with the automated guide; etc.), supplemental features from supplemental sources (e.g., inputs from therapy-associated users indicating participant's views towards the XR system), and/or any other suitable interaction-related features. Participant-virtual reality interaction models can be determined for participant interactions with a specific type of XR system, or component thereof (e.g., a specific headset, music track, or specific automated guide).

The inputs can include measurements of a participant's perceptual experience based on body movements (e.g., in response to a conversation or immersion element), emotions experienced by the participant; the content/subject matter the participant is looking at, imagining, or remembering (reconstructed in generative video form, image form, and by keyword descriptions); and sounds the participant hears (reconstructed in generative audio form, and by keyword descriptions). The XR system can be configured to track the participant's emotional responses, such as awe responses at behavioral and/or physiological level. For example, a participant's behavior can be measured in real time during the experience, using motion-tracking device systems. These devices allow tracking of head-movements, upper-limb and hands and facial-movements, as well as measuring posture. This information could be used to analyze, for example, non-verbal displays of awe. Psychophysiological responses can be assessed by collecting biofeedback such as one or more of electrical activity in the brain (EEG) and/or muscle (EMG), skin conductance (e.g., galvanic response), heart rate, skin temperature, and respiration, of other biofeedback, while the participant is exposed to stimuli, delivered through the XR medium or as a psychedelic experience. These responses can generate or refine an interaction plan for preparing for a psychedelic experience or identifying results consistent with a psychedelic-induced mystical-type experience experienced during a session. For example, biofeedback responses can be used to develop a musical accompaniment (e.g., playlist) configured to elicit a desired emotional response before, during or after a psychedelic experience. In some cases, the music can accompany portions of more than one stage. For example, a playlist can be configured to build an emotional response spanning the duration immediately before the psychedelic agent is taken to around the time the participant is experiencing the peak psychedelic effects and to diminish as the experience comes to an end. In other cases, multiple playlists are compiled for specific intervals within the psychedelic experience. Objective biofeedback responses to various music can be collected during the preparation stage. Reponses can be collected from a specific participant or a population of participants to be correlated with specific lyrics, songs, genres, BPM, etc. The evocative properties of any type of musical genres can be assessed (e.g., electronic vs instrumental, instrumental vs w. lyrics, etc. known to PHOSITA).

Inducing awe during a preparation stage may promote a mindset that facilitates achieving a mystical-type experience after taking a psychedelic agent. For example, an XR-induced awe experience can contribute to establishing a set and setting that can amplify the susceptibility and responsiveness of a participant to suggestions having the potential to alter the contents of consciousness, magnify the meaning a participant brings to the experience and influence a participant's perception, sensation, cognition, emotion, and behavior during a psychedelic experience.

Embodiments of the present disclosure can use an adaptive method to identify/assess the degree of a participant's awe using collected metrics, and adjust the virtual reality experience dynamically at runtime, to accommodate the use with a therapeutic psychedelic experience. Experiencing awe is associated with transformative changes at both psychological and physical levels. A participant can be presented with the set of prototypical awe-eliciting content during the preparation stage. The participant views the content and rates their experience/preference for of that content. Examples of awe-inducing content can include real-world and imagined environments. In some cases, the content includes fully immersive natural scenes of forest, mountains, and the Earth as viewed from deep space with the environmental sounds consistently within the virtual landscapes. The response can be calibrated against neutral environments (e.g., less likely to induce awe, such as a natural scene of green grass with few flowers and trees). In some cases, awe-enhancing content can include augmented reality elements such as the appearance of icons evoking religious, nostalgic, inspirational, or other meaningful associations for the participant. The emotional response to visual stimuli can be assessed by collecting biofeedback as discussed above for different auditory stimuli.

During an inquiry or preparation stage, the method of the present disclosure can include automatic and dynamic calibration and adaption of a virtual reality configuration to accommodate a participant's tolerances and comfort to promote a set and setting that is conducive to a therapeutic psychedelic experience. The calibration and/or adaptation can be based on biofeedback elicited by visual, auditory, and/or olfactory stimulus and/or self-reported preferences. For example, an initial configuration based on a participant's self-reported preference for a genre of music and/or visual stimuli can be adjusted based on biofeedback. In some cases, the preparation stage can include exposing the participant to a selection of immersive stimuli, alone or in combination to collect biofeedback, to establish baselines and thresholds for objective psychophysiological responses. A participant's subjective preference for an immersive stimulus can be indicated by a “Yes/No/Hard to say” response, which can be correlated with the objective measures. An initial selection of stimuli can be based in part on self-reported preferences and further include auditory and/or visual stimuli outside of the preferred genre(s) which are known to promote a therapeutic psychedelic experience in other participants. The initial selection can be refined based on biofeedback and/or subjective responses indicative of readiness for a therapeutic psychedelic experience.

In at least one embodiment, sensor feedback may be used to identify whether a participant is comfortable with a virtual reality experience, and based on the readings, automatically adjust various configuration settings of the virtual reality platform. Some feedback may be automatically collected by various sensors, and some feedback may require direct participant input. In some cases, a pre-set calibration may be performed or be customized for various specific or generic display devices or environments. Physical or virtual knobs may be present to allow the participant to adjust 3D levels, brightness, contrast, audio volume in preparation for a psychedelic experience. Automatic and dynamic calibration of parameters of the virtual reality experience can enable generation of participant-specific profiles that accommodate a participant's tolerances and limitations. Profiles may include identification of devices and environmental characteristics so that the virtual reality experience is automatically adjusted based on where the participant is, and which device they are using for the experience.

Some people are highly prone to motion sickness, and virtual reality immersion may exacerbate this tendency and contribute to discomfort after a participant takes a psychedelic agent. Embodiments of the present disclosure can use an adaptive method to calibrate a participant's tolerance using collected metrics, and adjust the XR experience dynamically at runtime (e.g., shift from VR to AR stimuli), to accommodate the use with a therapeutic psychedelic experience. A participant can be presented with the set of contents during the preparation stage for each tolerance metric to be tested. Each content within a set may vary in severity and range from most tolerable to least tolerable. The participant views the content and rates their tolerance of that content experience. For instance, a motion tolerance test may show content where objects move at varying speeds (e.g., slow to fast), and record the user's feedback regarding tolerability of the varying speeds presented in the content. The participant is tested on, and provides ratings for, a set of predetermined metrics that affect the XR experience. As new experiences are created and more feedback is received additional content to measure other metrics may be included. Feedback may be as simple as good, acceptable, or unacceptable, or more detailed such as a scale of 1-10. The results can be stored as part of the participant profile. Some tests can be automatic and provide tolerance results without explicit participant feedback.

Refinement of the participant model can contribute to validation/authentication functions of the method, such that one or more substantially permanent features of the participant can be used to verify the identity of the participant, in cooperation with sensing functions of the XR system. In variations, the sensing functions can be used to automatically verify the identity of the participant with whom the automated guide is interacting (e.g., based on speech patterns, voice recognition, vocalizations, motion behavior, facial recognition, etc.). The automated guide can prompt the participant to respond to verification questions based on the participant model.

Embodiments of the present disclosure can include determining participant goals for themselves. The participant goal can be directed to therapy in general or to a specific session. In some cases, a participant's session goal can include paying attention and remaining open to all experiences is helpful. Goals associated with the participant can include a medical goals category associated with more general health-related goals of the participant. Exemplary goals can include therapy adherence (e.g., to smoking cessation or diet plan), symptom management, physical wellbeing, mental wellbeing, and/or any other suitable medical goal. The interaction goals category can include goals for content and tone of outputs of the automated guide based on the participant model (e.g., a participant-automated guide interaction model), including but not limited to goals for improving interaction features, a goal for the automated guide to have desired (e.g., participant-defined) amounts of one or more of empathy, politeness, motivational tone, celebratory tone, sharing tone, encouraging tone, informing/educating tone, warning tone, warnings, entertaining tone, an anticipatory tone (e.g., in relation to stages of the psychedelic experience of the participant) and/or other suitable tones.

The medical goals of the participant can be refined over time based on new direct and indirect inputs informative of adverse or negative medical events (e.g., adverse effects of a treatment or medication, worsening of a medical condition), progress in relation to a medical condition (e.g., predicted physical wellbeing progress based on trends in physical condition, etc. predicted mental wellbeing progress based on trends in mental health condition, etc.), adherence behavior of the participant.

An interaction plan can include determining (e.g., generating, refining, etc.) an interaction plan having a conversation component and an XR component, based upon a participant model and/or a participant goal.

Outputs based on the most current models (e.g., refined models described above) are optimized for: 1) effectively engaging the participant and/or helping the participant achieve a therapeutic goal (e.g., the intention of the setting). An interaction plan (e.g., interaction model) can specify the manner in which an automated guide and/or XR system interact with a participant (e.g., through conversing with audio and/or visual content, etc.). For example, outputs for participant populations that are affiliated with a religious or spiritual group can include visual content depicting gods or goddesses in many traditions, angels or archangels in major traditions, spirit beings or spirit guides, power animals in shamanism, power objects in shamanism and other traditions, or other symbols of a participant's faith. The output can include visual and/or audio based on inputs, such as personal visual or audio files uploaded by the participant.

Conversation components and/or XR components for the interaction plan can specify content and/or tone to be expressed by the automated guide in conversing with the participant (e.g., including guiding prompts in response to different participant actions, etc.), but can additionally or alternatively specify one or more of: conversation scheduling (e.g., when to initiate conversation), participant goal data (e.g., participant goals to achieve with conversation components; historic success parameters for achieving participant goals for this participant and/or other participant with the conversation components; etc.), participant model data (e.g., the participant model data used in selecting the conversation component, etc.), metadata (e.g., versions; timestamps of when the conversation component was created; etc.), and/or any other suitable data. In examples, conversation components can include components (e.g., statements, questions, etc.) configured to convey personality aspects of the automated guide, configured to provide “small talk” interactions, and/or configured to help the participant improve or maintain psychedelic experience (e.g., mystical-type experience). The conversation components can be associated with a delivery format (e.g., textual form, graphical form, audio form, touch form such as braille, etc.) for the XR system to communicate with the participant based on the conversation component.

In a specific example, the output can include conversation components appropriate for the preparation stage. For example, a preparation stage conversation component can teach mindfulness techniques, such as diaphragmatic breathing with cognitive-behavioral stress management, or in a spiritual context, and/or hypnotic induction techniques. Additionally or alternatively, the conversation component can inform participants of what to expect emotionally and physically during a psychedelic experience generally, or based on the participant's stored treatment plan (e.g., what to expect for the specific psychedelic at the specific dose). The conversation component can address any specific hopes, fears or specific goals the participant has in respect to the upcoming experience and prepare the participant for emotionally intense thoughts, memories or experiences that may arise during the experience. The conversation component can present participants with instructions and food, alcohol or medication restrictions for the time starting 24 hours prior to a psychedelic-assisted psychotherapy session. The conversation component can prompt or encourage the participant sit or lie down and/or to select a playlist from a music library associated with the participant profile.

In another example, the output can include conversation components appropriate for the psychedelic experience stage. If inputs indicate a participant is exhibiting signs of psychological distress or panic the conversation component can remind the participant that he or she has taken a psychoactive drug and that he or she can stay with and work through the anxiety. The conversation components can offer support and reassurance, and/or remind the participant of the relaxation/mindfulness techniques learned during the preparation stage, to surrender to the experience, and/or to let go of resisting. If the participant is experiencing extreme distress or a psychotic response that does not resolve with conversational components, the output can include alerting the primary guide and/or initiating telecommunications with the primary guide.

Determining an interaction plan can include generating and/or executing an interaction plan model including any one or more probabilistic properties, heuristic properties, deterministic properties, and/or any other suitable properties. For example, interaction plans can include selecting interaction objects from a list of interaction objects to provide to the participant, using the XR system. Generating an interaction plan can include generating and/or applying one or more interaction trees. The interaction tree can include conversation flows with associated actions (e.g., augmented or immersive elements, initiating teleconference calls, activating supplementary devices, etc.) that are intended to be carried out by the XR system in relation to one or more participants. Generating the interaction tree can include pulling specific conversation components (e.g., sentences, questions, statements, phrases, etc.) from a conversation database, where each conversation component is associated with a branch of the interaction tree. Additionally or alternatively, conversation components can be generated with artificial intelligence (e.g., using artificial intelligence implementing modules configured to perform a selection process among different existing interaction trees or interaction subtrees).

Generating a tree that includes a set of nodes and one or more branches (i.e., downstream branches, upstream branches) can include associating each node with a logic condition (e.g., different potential participant responses to content to be expressed by an XR system or automated guide when reaching a particular node), and each branch is associated with a conversation component and/or augmented or immersive element component configured to be executed by the XR system. The logic condition of a node is configured to promote an improved therapeutic psychedelic experience interests of achieving the therapeutic goal (e.g., by achieving a mystical-type experience).

Generating an interaction tree can include associating one or more nodes with at least one set of potential conversation components and at least one set of potential augmented or immersive element components from which to respectively select conversation components and/or augmented or immersive element components for expressing content differently (e.g., different potential sentences and/or digital elements for communicating a greeting to the participant), tone (e.g., different words and/or digital elements used in expressing empathy to the participant), and/or other suitable interactions. The interaction tree can include branches connected to one or more nodes, where each branch can be associated with a different user response to content corresponding to the one or more nodes. Selecting one or more conversation components (e.g., from a set of conversation components available at a node) and/or augmented or immersive components (e.g., from a set of augmented or immersive components available at the node) is preferably based on one or more participant models (e.g., participant-automated guide interaction models) and/or participant goals.

An early interaction stage between the automated guide and the participant (e.g., during the preparation phase of a therapeutic psychedelic experience) configured to improve the therapeutic outcome can be used to configure the interaction tree. For example, interaction between the automated guide and the participant can be prioritized and, with a hypothesis that the participant responds strongly to sympathy, the node and associated branches of a interaction tree can include logic and conversation and/or augmented or immersive element components configured to show that the automated guide is sympathetic to the participant's condition. In another specific example, for a later stage of the therapeutic psychedelic experience, a treatment goal of the participant can be prioritized and, with a hypothesis that the participant responds strongly to encouragement, the node and associated branches of a interaction tree can include logic and conversation/augmented/immersive element components configured to encourage surrender during the psychedelic experience.

A system configured to implement a therapeutic psychedelic experience can include a set of computer-implemented rules defining the interaction plan as a function of one or more variables derived from one or more of a participant model (e.g., participant-automated guide-interaction model), a participant goal (e.g., therapeutic goal, etc.), supplementary device data, therapy-associated user data and/or any other suitable data. Computer-implemented rules can specify the types of variables to incorporate into selecting conversation components and/or augmented or immersive element components, weights to assign to different variables, standardization units, processing operations (e.g., data normalization, filtering, averaging, combining, etc.), and/or any other suitable aspect in relation to applying computer-implemented rules for determining an interaction plan.

In some case, the system can include generating and/or applying one or more interaction plan machine learning models employing machine learning algorithm(s) that can be characterized by a learning style including any one or more of: supervised learning (e.g., using logistic regression, using back propagation neural networks), unsupervised learning (e.g., using an Apriori algorithm, using K-means clustering), semi-supervised learning, reinforcement learning (e.g., using a Q-learning algorithm, using temporal difference learning), and any other suitable learning style. Furthermore, the machine learning algorithm can implement any one or more of: a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and/or any suitable form of machine learning algorithm. In a specific example, the learning model can include training a neural network model (e.g., a generative neural network model without predetermined conversation and/or augmented or immersive element components) with an input neural layer using features derived from one or more participant models, participant goals, content and/or tone expressed by the participant, primary guide, and/or automated guide up to the present time in a current conversation, and/or any other suitable data, where the neural network model can dynamically output conversational components, augmented or immersive element components, and/or any other suitable information associated with an interaction plan.

In some cases, generating and/or executing different interaction plan determination models (e.g., different types of conversation trees; conversation trees versus machine learning models; etc.), includes using different interaction plan models for different participants (e.g., different individual participants, different participant populations, etc.), different automated guides and/or XR systems (e.g., different types of systems possessing different sets of sensors), and/or can be applied in different manners based on any suitable criteria. For example, the method can include generating a first set of conversation trees (e.g., including more nodes associated with empathy tones) for a first participant population (e.g., dementia patients), and generating a second set of conversation trees (e.g., including more nodes associated with achieving therapeutic goals, such as nodes including content for asking about a traumatic experience for a second participant population (e.g., participants experiencing PTSD).

Methods of the present disclosure can include determining any number of interaction plans for any number of users (e.g., participants, therapy-associated users, etc.). A given interaction plan can be used by any suitable number of automated guides and/or XR systems, and an automated guide/XR system can execute any suitable number of interaction plans. In examples, determining an interaction plan for a first participant can be based on interaction plans for other participants (e.g., efficacy of interaction plans in achieving a mystical-type experience).

In some cases, methods of the present disclosure include generating an analysis of efficacy of a first interaction plan for achieving a first therapeutic goal associated with a first participant; generating a second interaction plan for achieving a second therapeutic goal associated with a second participant, based on the analysis; and executing the second interaction plan with a second automated guide/XR system associated with the second participant.

In a variation, methods of the present disclosure can include generating a interaction plan for guiding communication between two or more XR systems. These interaction plans can specify any one or more of: communication protocols (e.g., wireless communication protocols between systems; protocols for communicating with supplementary devices; etc.), software update transfers, participant data transmission (e.g., user inputs, participant models, participant goals, associated interaction plans, etc.), conversation and/or augmented or immersive element components for interaction between automated guides/XR systems and/or any other suitable information. In a specific example, a method can include determining a system interaction plan specifying a primary/secondary framework between a primary virtual reality and one or more secondary XR systems; transmitting a set of interaction plans from the interaction engine to the primary system (e.g., through WiFi); and distributing the set of interaction plans from the primary system to one or more secondary systems (e.g., through Bluetooth Low Energy). The method can include generating an interaction plan for guiding communication between one or more automated guides/XR systems and one or more supplementary devices (e.g., user device, medical device, etc.).

Methods of the present disclosure include generating interaction plans for guiding communication between one or more automated guides and one or more therapy-associated users such as a care provider or therapist overseeing the psychedelic experience.

In some cases, determining a participant model and/or determining a participant goal be performed in response to and/or concurrently with another trigger condition (e.g., analyzing efficacy of an interaction plan; determining a participant-automated guide engagement level below a threshold condition; etc.), performed at predetermined time intervals, and/or performed with any suitable temporal relationship to the psychedelic experience. Refining (e.g., updating) the interaction engine (e.g., over time) can improve the accuracy of the interaction engine (e.g., thereby improving the functionality of computer-related technology) in generating interaction plans with conversation components and augmented or immersive element components tailored to achieving participant goals. Refining the interaction engine over time can include comparing planned conversations between the participant and the automated guide to actual conversations between the participant and the automated guide, and determining efficacy of the planned conversations in terms of experience outcomes. In a specific example, a planned conversation intended to facilitate relaxation before taking of a psychedelic by the participant can be tested, and the actual conversation can be used to refine future conversations for facilitating relaxation. The method can include determining an interaction schedule specifying the timing for expressing conversation components in relation to expressing augmented or immersive element components.

The one or more interaction plans, associated conversation components, associated augmented or immersive element components, and/or other elements can be stored in an interaction database (e.g., as part of a data store of the system). The conversation components and/or augmented or immersive element components can be human crafted; however, one or more elements of the interaction database can additionally or alternatively be non-human crafted (e.g., automatically generated using artificial intelligence to craft different aspects of conversations for achieving specific goals). In a variation, generating conversation components and/or augmented or immersive element components with the interaction engine can involve using (initially) human-crafted conversation components and learning from responses to those conversation components using appropriate machine learning algorithms, in order to generate subsequent AI-crafted conversation components for engaging the participant.

Each conversation and/or augmented or immersive element component of an interaction can be tagged (e.g., with a participant goal), such that the appropriate interaction components can be selected (e.g., for achieving participant goals) from the conversation/augmented/immersive element database for designing the interaction between the automated guide and/or XR system and the participant. In specific examples, an interaction component (e.g., conversation component, augmented or immersive element component, etc.) can be tagged as one or more of: “empathetic”, “sympathetic”, “polite”, “motivational”, “celebratory”, “sharing”, “encouraging”, “informing”, “educating”, “warning”, “reminding”, “entertaining”, “anticipatory”, and/or any other suitable tone-associated tag.

Executing the interaction plan with the automated guide/XR system, thereby promoting engagement and improving the therapeutic psychedelic experience, and therapeutic outcome for the participant. The interaction plan can be enacted through an output device (e.g., speaker, graphical display), actuatable elements, and/or other suitable components of the XR system in order to engage the participant and elicit further interactions between the participant and automated guide. The executed interaction plan(s) can be determined and transmitted (e.g., from the interaction engine) to one or more XR systems, but interaction plans can be received by XR systems in any suitable manner. Executing the interaction plan preferably includes outputting a communication (e.g., conversational audio; updated conversational audio based on an updated interaction plan; etc.) based on one or more conversation components, and/or other action (e.g., the action of an immersive element). Outputting a communication and/or initiating an augmented or immersive element can be coordinated and/or performed based on an interaction schedule. The method can include transmitting commands (e.g., which can be included in an interaction plan) from a control model of the interaction engine/XR system to output elements (e.g., actuators, display modules, speakers, etc.) of the system, or to drive communication of the automate guide in engaging the participant with speech. Transmitting commands include driving touch display components of the system to allow a participant to engage with a touch screen in providing responses to queries, or interacting with the system using one of a set of preselected responses provided at the touch screen.

Methods of the present disclosure include activating and/or controlling a supplementary device in facilitating participant engagement, participant goals, and/or in any other suitable purpose. Supplementary devices can include any one or more medical devices (e.g., a biosignal detector, fitness wearable, cardiovascular device, head-mounted wearable device, wrist-mounted wearable computing device, etc.), a user device (e.g., smartphone, laptop, desktop computer, tablet, smart watch, toys, etc.), chatbot-enabled devices, additional XR systems, and/or any other suitable devices. Activating a supplementary device is preferably performed with an XR system (e.g., through a wireless communication channel between the XR system and the supplementary device), but can additionally or alternatively be facilitated by a device controlling engine (e.g., remote computing system; affiliated with the interaction engine; same as the interaction engine; etc.) and/or any other suitable entity. Controlling supplemental devices is preferably performed substantially concurrently with presenting communications and/or augmented or immersive elements with the automated guide/XR system, but can be performed with any suitable temporal relationship (e.g., serially, in parallel) with executing any portion of one or more interaction plans. Activation and control can include generating and/or transmitting a notification to a supplementary device, retrieving data from the supplementary device, determining control instructions for controlling the supplementary device, and/or any other suitable operations.

The method can include initiating telecommunication between a participant and a therapy-associated user with the automated guide, which functions to use the XR system to facilitate a communication channel with therapy-associated user such as a care provider, therapist, physician, etc.) to allow the participant to communicate with the therapy associated-user. Initiating telecommunication can be an action specified in an interaction plan. In an example, the method can include initiating telecommunication substantially concurrently with expressing a conversation component (e.g., “Let me get your doctor for you”) and/or an augmented or immersive element component (e.g., providing calming visual or musical cues)

The method can include any other steps configured to increase engagement with the XR system to facilitate the therapeutic psychedelic experience and/or to achieve therapy-related goals of a participant. Portions of the method can be performed in serial (e.g., in response to, etc.), parallel (e.g., concurrently on different threads for parallel computing to improve system processing ability for determining and/or executing interaction plans, etc.), and/or with any suitable temporal relationship.

Systems of the present disclosure for implementing a therapeutic psychedelic experience includes: a XR system operable to execute an interaction plan (e.g., interaction model) for communicating with the participant and a therapy-associated user. The XR system includes an input device operable to receive user inputs from the participant, an output device operable to present a communication to the participant based on a conversation component of the interaction plan, and an actuatable element operable to initiate an augmented or immersive element of the interaction plan; and an interaction engine (e.g., a remote computing system) operable to determine a participant model (e.g., including a participant-automated guide interaction model, a personality model, a mood model, a biographical model, a medical model, etc.) for the participants based on the inputs, determine an experience goal for the participant based on the inputs, and generate the interaction plan based on the participant model and/or the experience goal. The system can include one or more supplementary devices, data stores, holographic entities, and/or any other suitable components. Any components of the system are configured to implement at least a portion of the method.

The system can include one or more interaction engines configured to process inputs related to the method, in order to output interaction plans (e.g., with conversation and augmented or immersive element components) operable to be implemented using one or more XR systems. The interaction engine can be implemented in one or more of: a remote server, in the cloud, a computing system of the XR system (e.g., a processing system encapsulated within a housing of the system), in a personal computing system, in a computing system of a mobile device carried by the participant (e.g., a smartphone, tablet, wrist-mounted mobile computing device, head-mounted wearable computing device, etc.), where the computing executes instructions for refining models and/or executing interaction plans according to the method.

The interaction engine and/or one or more XR systems can generate, store, and/or retrieve profiles for different users (e.g., participants, care providers, etc.). User profiles are preferably associated with one or more participant models, participant experience goals, interaction plans, user identifiers (e.g., user account credentials, biometric credentials such as facial recognition patterns, etc.). In examples, a single interaction engine can execute different interaction plans for different users, such as based on the user profiles for the different users. The interaction engine can retrieve user profiles in response to recognizing users who are interacting with the XR system/automated guide, where recognition can be from any one or more of: biometric recognition (e.g., facial recognition, voice recognition, thumbprint recognition, etc.), receiving user account credentials, conversation (e.g., receiving a response to a question of “Who am I speaking to today?”, etc.), and/or any other suitable form of recognition. In a specific example, the XR system can be operable to collect an optical dataset of a user with an optical sensor; recognize the user (e.g., a participant out of a plurality of participants) based on the optical dataset; and output a communication based on an interaction plan generated for the recognized user. In another specific example, the interaction engine and/or XR system can be operable to recognize a therapy-associated user (e.g., a care provider, sitter); and retrieve an interaction plan for the therapy-associated user based on a corresponding user profile, where the interaction plan can include conversation, augmented and/or immersive components for facilitating assistance by the therapy-associated user in achieving an experience goal of the participant, and where the interaction plan and/or associated components can be selected based on a user model (e.g., a relationship model specifying engagement between the therapy-associated user and the automated guide a care provider model; etc.). However, user profiles associated with different users can be configured in any suitable manner.

The system can include an XR system and/or automated guide operable to execute an interaction plan. The XR system and/or automated guide functions to engage a participant using one or more conversation components, augmented and/or immersive element components of an interaction plan in order to, for example, achieve one or more participant goals (e.g., psychedelic experience goals). The XR system can include one or more: input devices; output devices; actuatable elements, processing systems; a communication module (e.g., wired; wireless; for transmitting and/or receiving data with an interaction engine, supplementary devices, and/or other suitable components; etc.), a housing (e.g., defining the visual appearance of the XR system), and/or any other suitable components. Output devices include speakers, displays, holographic displays, touch elements such as braille generators, etc.) configured to enable the XR system to drive interactions with the participant. Input devices can include any one or more of: optical sensors, touch sensors such as capacitive touch sensors, proximity sensors for sensing the position of the participant such as through radar-based sensors, location sensors such as GPS systems, motion sensors such as accelerometers and gyroscopes, audio sensors such as microphones, touch screens, keypads, keyboards, mice, joystick, and/or other suitable components that enable and/or facilitate participant interaction with the XR system and/or automated guide. One or more portions of the interaction engine described above can be integrated with the XR system; however, in alternative variations, the XR system can serve as a conduit for interaction (e.g., through the automated guide, input devices, and output devices) with the participant, with computing systems implemented in components distinct from the XR system.

In a variation, the XR system can include one or more optical sensors (e.g., cameras, light sensors, etc.) operable to capture optical datasets of the participant, of therapy-associated users, and/or of any suitable entity. Optical datasets can be used for: object classification (e.g., recognizing users, associated objects, environmental settings, supplementary devices, etc.), position determination (e.g., position of participant), emotion recognition (e.g., based on captured facial expressions, which can be used independently and/or along with other suitable data for determining models such as user-automated guide relationships, etc.). In another example, the optical sensor can track a participant (e.g., keep a participant in the field of view of the optical sensor). In a specific example, the XR system (and/or other component of the system) can include a processing system operable to determine a position of a user (e.g., participant, therapy-associated user) relative to the XR system on an optical dataset captured by the optical sensor and to actuate an augmented or immersive element.

The system can include a data store that functions to store and/or transmit outputs and/or inputs of the method for use by models (e.g., participant models, etc.) and/or other elements (e.g., interaction plans, etc.) implemented according to the method. The data store can be implemented in hardware components (e.g., in servers, in computing systems, in the XR components (e.g., HMD), etc.) and/or in the cloud, and can store data from a single participant and/or from a population of participants. The refinement process described above can include processing of inputs from a population of individuals, whose data is stored in and transmitted from the data store described above.

In some cases, the automated guide is a holographic entity that functions to enable non-physical interactions with a participant and/or an animated entity that the participant can interact with using a display. Additionally or alternatively, the automated guide can be non-holographic entity (e.g., audio only).

The Examples illustrate the architecture, functionality and operation of possible implementations of systems, methods and computer program products according to preferred embodiments, example configurations, and variations thereof. In this regard, each block in the flowchart or block diagrams may represent a module, segment, step, or portion of code, which includes one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions. The embodiments include every combination and permutation of the various system components and the various method processes, including any variations, examples, and specific examples.

The method and/or system of the present disclosure can be embodied and/or implemented at least in part as a machine configured to receive a computer-readable medium that stores computer-readable instructions. The instructions can be executed by computer-executable components that are integrated with the application, applet, host, server, network, website, communication service, communication interface, hardware/firmware/software elements of a user computer or mobile device, wristband, smartphone, or any suitable combination thereof. The instructions can be executed by computer-executable components integrated by computer-executable components, which are integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component is preferably a processor but any suitable dedicated hardware device can (alternatively or additionally) execute the instructions.

In some embodiments, the method of the present disclosure includes identifying participants likely to have a positive psychedelic-induced experience, likely to be non-responders, or who are likely have a non-therapeutic experience. A potential participant's susceptibility to a therapeutic experience or a mystical-type experience associated with psychedelic treatment can be determined by assessing and weighing specific predictors, e.g., inclusion or exclusion criteria for indicating whether a subject suffering from a psychological disorder is likely to respond to treatment with a psychedelic agent. Examples of predictors of a therapeutic response include a high degree of openness to absorbing and self-altering experiences (“openness”) and/or a low degree of cognitive resistance (resistance) and/or a high degree of identity distress (e.g., feelings of apprehension, worry, and emotional upheaval in regard to the inability to resolve a number of issues related to identity) and/or low measures of preoccupation and/or high measures of surrender and/or spiritual motivations, norm motivations, pleasure motivations, or betterment motivations.

### Mental Health Conditions

The method and/or system of the present disclosure can be configured or adapted for the treatment of addictions and substance abuse. For example, the methods of the invention can be used to treat drug addictions, such as addictions to recreational drugs or addictive medications, and to treat addictive behaviors, including food addiction, eating disorder, binge eating disorder, pathological gambling, pathological use of electronic devices, pathological use of electronic video games, pathological use of electronic communication devices, pathological use of cellular telephones, addiction to pornography, sex addiction, obsessive-compulsive disorder, impulse control disorder, compulsive spending, intermittent explosive disorder, kleptomania, pyromania, trichotillomania, compulsive over-exercising, and compulsive overworking. A participant in need of substance abuse treatment can misuse or develop a dependence on an addictive agent such as alcohol (e.g., ethanol), gamma hydroxybutyrate (GHB), caffeine, nicotine, cannabis (marijuana) and cannabis derivatives, opiates and other morphine-like opioid agonists such as heroin, phencyclidine and phencyclidine-like compounds, sedative hypnotics such as benzodiazepines, methaqualone, mecloqualone, etaqualone and barbiturates and psychostimulants such as cocaine, amphetamines and amphetamine-related drugs such as dextroamphetamine and methylamphetamine, or on an addictive medication such as benzodiazepines, barbiturates, and pain medications including alfentanil, allylprodine, alphaprodine, anileridine benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofenitanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, OXYCONTIN®, oxymorphone, papaveretum, pentazocine, phenadoxone, phenomorphan, phenazocine, phenoperidine, piminodine, piritramide, propheptazine, promedol, properidine, propiram, propoxyphene sufentanil, tramadol, and tilidine.

Systems and methods of the present disclosure can be configured or adapted for treatment of a psychological disorder that includes a repetitive body-focused behavior (e.g., a tic disorder, such as Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder).

Systems and methods of the present disclosure can be configured or adapted for the treatment of anxiety disorders, or unwarranted or inappropriate worry often accompanied by restlessness, tension, distraction, irritability and sleep disturbances. Anxiety disorders suitable for treatment can include generalized anxiety disorder (GAD), panic disorder, social anxiety, post-traumatic stress disorder (PTSD), acute stress disorder (ASD), obsessive compulsive disorder (OCD), and social phobias. Unwarranted or inappropriate worry can include worry about life events such as marital relationships, job performance, health, money, and social status.

Systems and methods of the present disclosure can be configured or adapted for the treatment of anxiety disorders and/or depressive disorders associated with life-threatening or terminal diseases and diminishing health due to the progression of a terminal disease, as part of a palliative care plan.

Systems and methods of the present disclosure can be configured or adapted for the treatment of a depressive disorder (e.g., major depression, melancholic depression, atypical depression, or dysthymia). The depressive disorder may be associated with one or more prodromal symptoms selected from the group consisting of depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses. The depressive disorder can be associated with a diagnosis with a terminal illness, a life-threatening illness, or cancer.

Systems and methods of the present disclosure can be configured or adapted for the treatment of a psychosomatic symptom or a somatic symptom (e.g., chronic pain, anxiety disproportionate to severity of physical complaints, pain disorder, body dysmorphia, conversion, hysteria, neurological conditions without identifiable cause, or psychosomatic illness).

The following Examples are intended to illustrate applications of the method and system described above and should not be construed as to narrow the scope of the disclosure. One skilled in the art will readily recognize many other ways in which the exemplary configurations could be applied. Numerous variations and modifications may be made while remaining within the scope of the invention.

### Examples

### Participant Preparation

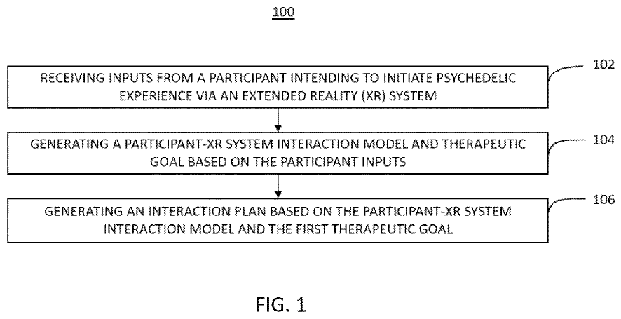

Referring to FIG. 1, a method 100 for preparing a participant profile for guiding an Extended Reality (XR)-assisted therapeutic psychedelic experience is provided. Method 100 can be initiated at step 102 by receiving inputs of a participant intending to initiate a psychedelic experience collected via an XR system at a remote interaction engine. The inputs can include biometric data for participant identification (e.g., image and/or voice print), information about the type and dose of psychedelic agent the participant plans to take, and participant data including personality traits, biographical information, medical history, and biofeedback. The inputs can be collected using a microphone, keyboard, mouse, wearable device such as brain activity sensors, muscle activity sensors, skin temperature sensors, heart rate sensors, respiratory rate sensors, eye movement sensors, bioimpedance sensors, and/or galvanic response sensors. The initial inputs can be received with minimal prompting/stimulus by the automated or primary guide to establish a first baseline. Threshold limits for psychophysiological responses can be established and stored at the remote interaction engine. The limits can be refined in response to conversational elements of an automated guide or a primary guide, and/or augmented or immersive components. Inputs can include establishing preferences and dislikes for digital elements of the augmented or immersive component elements (e.g., aspects of visual, auditory, olfactory, and/or tactical stimuli), preferences and dislikes for conversational components (voice of automated guide, tone of automated guide (“empathetic”, “sympathetic”, “polite”, “motivational”, “celebratory”, “sharing”, “encouraging”, “informing”, “educating”, “warning”, “reminding”, “entertaining”, “anticipatory”). Preference and/or dislike can be confirmed by objective measures (e.g., biofeedback). In some cases, the inputs are collected during a simulated psychedelic experience. For example, the participant's psychophysiological responses when immersed in a virtual environment that is known to be capable of inducing a mystical-type experience can be collected and stored in the remote interaction engine.

In step 104, the participant inputs are used to generate a participant-XR system interaction model and therapeutic goal for the psychedelic experience. The participant-XR system interaction model facilitates engagement by the automated guide to optimize set and setting, with minimal direct engagement by the primary guide.

In step 106, an interaction plan is generated and stored at the remote interaction engine. Execution of the interaction plan generates a virtual and/or augmented therapeutic environment (i.e., a XR environment optimized for achieving a therapeutic experience). The interaction plan will be executable by the XR-system when the participant is ready to initiate a psychedelic experience. The primary guide can review the interaction plan and revise as necessary at this step. In some cases, the interaction plan includes a plan for recording the experience (e.g., statements made by the participant during the peak experience) and/or collecting post-experience inputs for use during integration of the psychedelic experience.

In some embodiments, steps 102-106 are performed iteratively to refine the generated participant-XR system interaction model and/or interaction plan for a specific participant or to train an AI interaction engine.

In some embodiments, steps 102-106 are performed for two or more participants concurrently, and the inputs received by a single remote interaction engine to generate at least two participant-XR system interaction models and/or interaction plans that will be overseen by a single primary guide when the participants are ready to initiate the therapeutic psychedelic experience.

### Establishing Set and Setting

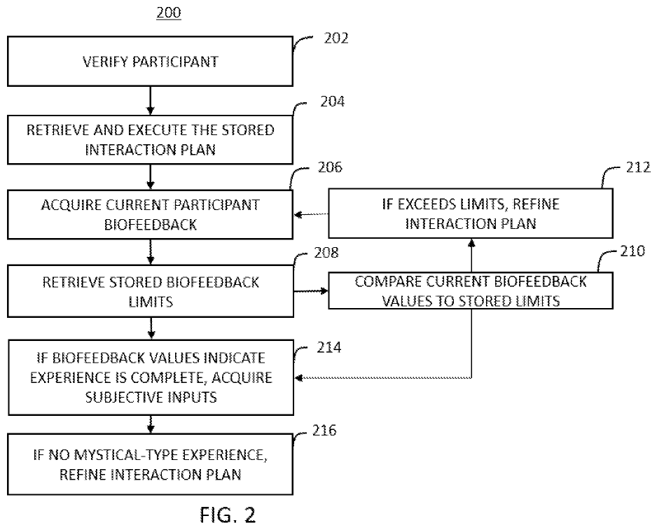

Referring to FIG. 2, a method 200 for establishing set and setting for a therapeutic psychedelic experience using a XR system is provided. Method 200 can be initiated at the XR system in connection with the remote interaction engine by a participant with a generated interaction plan (i.e., according to one or more embodiments of Method 100). In step 202, the method verifies the identity of the participant based on stored inputs of step 102 (e.g., biometric data or biographical data (e.g., username and password). In response, in step 204 the XR system retrieves and executes the participant's interaction plan. The one or more components of the XR system will engage the participant by generating an interaction tree of objects associated with a first set of conversation components and a first set of augmented or immersive element components for presenting content; and a set of sub-objects connected to the object, each connection associated with a different participant response to the content; and selecting the first conversation component from the first set of conversation components and the first augmented or immersive element component from the first set of augmented or immersive element components based on the participant-XR system interaction model. During the actuation of the first set of conversation and augmented or immersive elements, the XR system acquires current biofeedback inputs from the participant in step 206. At least one of the acquired inputs will be based on the same parameter as the inputs of step 102, (e.g., brain activity) and is indicative of a participant's degree of relaxation, openness, surrender, and acceptance of the treatment regimen and automated engagement tools. In step 208, the XR system retrieves the stored limits for the biofeedback parameter. The XR system compares the current value to the stored limit at step 210. If the current value is within acceptable limits, the method proceeds to step 214 to verify the psychedelic agent is the expected agent at the expected dose and to confirm that the participant wishes to have a psychedelic experience. After confirmation of the acceptance of the proposed experience, in step 216 the XR system communicates establishment of a therapeutic set and setting with the primary guide.

Returning to step 210, if the current value is outside the stored limits for the biofeedback parameter, the XR system engages with the remote interaction engine in step 212 to refine the interaction plan and/or the participant-XR interaction model. In some cases, the refined interaction plan generates a second set of conversation components and/or a second set of augmented or immersive element components for presenting content; and a set of sub-objects connected to the object, each connection associated with a different participant response to the content; and selects a second conversation component, or a second augmented or immersive element component based on the refined participant-XR system interaction model. For example, a second conversation component can include instructions for the automated guide to inquire about and address any specific fears, emotionally intense thoughts, memories or experiences that may have arisen in expectation of the psychedelic experience. A second augmented element component can include adjusting auditory stimuli and/or introducing an olfactory stimulus. During actuation of the second set of conversation and/or augmented or immersive elements, the XR system returns to step 206 to acquire current biofeedback inputs from the participant. Optionally the most recent biofeedback inputs are compared to determine if the values are improving. The system can compare the newly acquired values to the stored limits for the biofeedback parameter as performed previously (repeat step 210) and proceed step 212 or 214 based on the result. In some cases, the XR-system can be configured to communicate with the primary guide if the current parameter continues to exceed the stored limits after the first attempt to refine the interaction plan. The primary guide can recheck the out-of-range data. If the recheck confirms the parameter exceeds the stored limit, the primary guide may further refine the interaction plan to support the participant in managing stress, reluctance, or other negative feelings about taking the psychedelic agent until the parameter is within acceptable limits or recommend postponing the experience.

### Initiating and Monitoring Psychedelic Experience

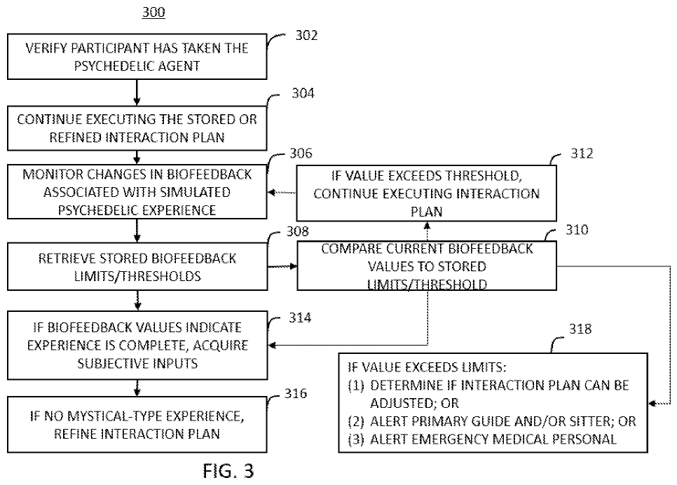

Referring to FIG. 3, a method 300 for initiating and monitoring a therapeutic psychedelic experience using a XR system is provided. Method 300 can be initiated at the XR system by a participant with established set and setting (i.e., according to one or more embodiments of Method 200). In step 302, the method verifies that the participant has taken the expected psychedelic agent at the expected dose. Verification can be based on direct interaction of the participant with the system, or based on indirect psychophysiological responses which are detected by biofeedback sensors. Upon confirmation, in step 304 the XR system continues to execute the participant's generated or refined interaction plan, in accordance with Method 200. The one or more components of the XR system will engage the participant by generating an interaction tree of objects associated with a set of conversation components and/or a set of augmented or immersive element components for presenting content while the effects of the psychedelic are being experienced based on a participant-XR system interaction model defined by inputs received during a simulated psychedelic experience. During the actuation of the set of conversation and/or augmented or immersive elements, the XR system monitors changes in biofeedback associated with the simulated experience in step 306. For example, changes in brain activity which are detectable by EEG can indicate that the participant is experiencing a dream-like state. In addition, the XR system can monitor biofeedback indicative of intense emotional responses. In step 308, the XR system retrieves the stored limits for the biofeedback parameter(s). The XR system compares the current value to the stored limit/threshold at step 310. If the current value of the simulated psychedelic biofeedback parameter is below expected threshold, the result can be indicative of the end of the psychedelic experience, method can proceed to step 314 to acquire subjective inputs about the experience from the participant. For example, the XR system can be configured to present and receive vocal responses to one or more of the “Mystical Experience Questionnaire” (MEQ30), Beck Depression Inventory, “Questionnaire for recording extraordinary states of consciousness' (5D-ABZ), “Altered States of Consciousness Rating Scale”, “Phenomenology of Consciousness Inventory”, “Hallucinogen Rating Scale” (HRS 3.06), “Addiction Research Inventory”, “States of Consciousness Questionnaire”, “Persisting Effects Questionnaire”, “Psychotomimetic States Inventory”, “Ego Dissolution Inventory (EDI)”, “Hood Mysticism Scale”, “Mystical Experience Questionnaire (MEQ43)”, “States of Consciousness Questionnaire” (SOCQ), and “Persisting Effects Questionnaire”. After receiving the subjective inputs, method 300 can include analyzing the efficacy of the interaction plan to improve the likelihood that the participant would have a mystical-type experience. In step 316 the XR system can refine the interaction plan based on the subjective inputs.

Returning to step 310, if the current value is above the stored threshold for the biofeedback parameter associated with the simulated psychedelic experience, in step 312, the XR system continues to execute the interaction plan. If a biofeedback limit that indicating an adverse medical event is exceeded, the XR-system is configured to grade the severity of the event. In some cases, the XR-system can adjust the interaction plan to assist with calming the participant. Alternatively or additionally, the XR-system can alert the primary guide or a sitter of the adverse medical event. In some cases, the XR-system contacts emergency medical personnel (e.g., signs of cardiac arrest, shock, psychotic break, physical injury while under the influence of the psychedelic agent. If the XR-system (e.g., automated guide), primary guide or sitter deescalated the event such that the biofeedback parameter returned to acceptable levels, the XR system can return to step 306 to monitor changes in biofeedback inputs from the participant. The system can compare the newly acquired values to the stored limits for the biofeedback parameter as performed previously (repeat step 310) and proceed step 312, 314, or 318 based on the result. If a mystical-type experience is reported, the integration stage can utilize the interaction plan to re-immerse the participant in the therapeutic experience. All conversation and augmented/immersive components of the plan can be replayed separately (e.g., the musical accompaniment during the peak experience alone) or in combination (e.g., musical and olfactory stimuli during the early stages of the experience).

Other embodiments of the present disclosure are possible. Although the description above contains specific examples, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above. Rather, the scope of the present disclosure fully encompasses other embodiments which may become obvious to those skilled in the art,

The scope of this disclosure should be determined by the appended claims and their legal equivalents. Reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

What is claimed is:

1. A system for an Extended Reality (XR)-guided therapeutic psychedelic experience comprising:
an XR system operable to execute a first interaction model for communicating with a first participant of a psychedelic experience, the XR system comprising:
an input device operable to receive inputs from the participant;
an output device operable to present a communication to the participant based on a first conversation component of the first interaction model; and
an actuatable element operable to present an augmented or immersive element based on an augmented or immersive element component of the first interaction model; and
an interaction engine operable to:
determine a participant-XR system interaction model associated with engagement between the participant and the XR system, based on the participant inputs;
determine a first therapeutic goal associated with the participant, based on participant inputs; and
generate the first interaction model comprising the first conversation component and the augmented or immersive element component based on the participant-XR system interaction model and the first therapeutic goal.

2. The system of claim 1, wherein the input device comprises a biofeedback sensor.

3. The system of claim 2, wherein the biofeedback sensor is selected from the group consisting of brain activity sensors, muscle activity sensors, skin temperature sensors, heart rate sensors, respiratory rate sensors, eye movement sensors, bioimpedance sensors, and galvanic response sensors, or a combination thereof.

4. The system of claim 2, further operable to execute a primary guide interaction model for communicating with a primary guide supporting the participant based on biofeedback, wherein the output device is further operable to present communication to the participant from the primary guide and the actuatable element is further operable to present augmented or immersive elements based on an augmented or immersive element component of the primary guide interaction model.

5. The system of claim 1, wherein the interaction engine is further operable to determine a participant model comprising at least one of the following: personality traits of the participant, emotional states of the participant, biographical information of the participant, and a medical history of the participant; and wherein the first interaction model is further based on the participant model.

6. The system of claim 5, wherein the input device is operable to receive at least one of the following inputs from or about the participant: biometric data, biographical information, relationship information, demographic information, preferences and dislikes for digital elements of the augmented or immersive component elements, preferences and dislikes for conversational components, musical tastes, religious or spiritual affiliation, and location.

7. The system of claim 1, wherein the interaction engine is operable to:
generate the first interaction model using an interaction tree comprising:
an object associated with a first set of conversation components and a first set of augmented or immersive element components for presenting content; and
a set of sub-objects connected to the object, each connection associated with a different participant response to the content; and
select the first conversation component from the first set of conversation components and the first augmented or immersive element component from the first set of augmented or immersive element components based on the participant-XR system interaction model.

8. The system of claim 1, further comprising at least one additional XR system to execute at least one additional interaction model for interacting with a second participant of a second psychedelic experience, or a plurality of psychedelic experience participants, the at least one additional interaction model comprising at least one additional conversation component and at least one additional augmented or immersive element component tailored to the second participant or each of the plurality of participants based on a at least one additional participant-XR system interaction model and at least one additional therapeutic goal.

9. The system of claim 8, wherein the at least one additional XR system further comprises a biofeedback sensor.

10. The system of claim 9, further operable to execute a primary guide interaction model for communicating with a primary guide supporting the second participant based on biofeedback, wherein the at least one additional XR system is further operable to present communication to the second participant or the plurality of participants from the primary guide and the actuatable element is further operable to present augmented or immersive elements based on an augmented or immersive element component of the primary guide interaction model.

11. A method of improving a therapeutic outcome of a psychedelic experience comprising:
receiving, at a remote interaction engine, inputs of a first participant of a psychedelic experience collected at a first XR system in response to outputting conversational audio for the first participant at a speaker of the first XR system;
refining, at the remote interaction engine, a first participant-XR system interaction model based on the first participant inputs;
refining, at the remote interaction engine, a first therapeutic goal based on the first participant inputs;
generating a first interaction plan comprising a conversation component, based on the first participant-XR system interaction model and the first therapeutic goal;
transmitting the first interaction plan from the remote interaction engine to the first XR system; and
outputting, at the speaker of the first XR system, updated conversational audio based on the conversation component of the first interaction plan.

12. The method of claim 11, wherein the first participant inputs include psychedelic agent data and biofeedback.

13. The method of claim 12, wherein biofeedback includes at least one of the following parameters: speech pattern, brain activity, respiratory rate, heart rate, muscle activity, electrodermal, skin temperature, eye movement tracking, and motion detection.

14. The method of claim 12, wherein refining first participant-XR system interaction model comprises:
sensing a current value for a biofeedback parameter of the first participant and comparing the current value with stored biofeedback parameter limits of the first participant; and
communicating the current value of the first participant biofeedback parameter to a primary guide through the XR system if the current value is outside the stored limits; or
retransmitting the first interaction plan from the remote interaction engine to the XR system if the current value is within the stored limits.

15. The method of claim 11, further comprising:
generating an analysis of the efficacy of the first interaction plan for achieving the first participant therapeutic goal;
generating, at the remote interaction engine, a second interaction plan for achieving a second participant psychotherapy goal associated with a second participant of a second psychedelic experience, based on the analysis; and
executing the second interaction plan with a second XR system associated with the second participant.

16. The method of claim 15, wherein generating the analysis includes receiving responses to a questionnaire completed by the first participant after the psychedelic experience.

17. The method of claim 15, further comprising:
receiving, at the remote interaction engine, second participant inputs collected at the second XR system in response to executing the second interaction plan for the second participant;
determining a second participant-XR system interaction model based on the second participant inputs and the first participant-XR system interaction model; and
updating the second interaction plan based on the second participant-XR system interaction model.

18. The method of claim 11, further comprising:
determining a participant model comprising a personality model associated with at least one of the group consisting of personality traits of the first participant, a mood model associated with emotional states of the first participant, a biographical model associated with contextual information of the first participant, and a medical model associated with a medical history of the first participant, and the first participant-XR system interaction model; and
determining a therapeutic goal, based on the first participant inputs,
wherein generating the first interaction plan is further based on the participant model.

19. The method of claim 11, further comprising:
receiving primary guide inputs associated with the psychedelic experience of the first participant; and
determining a primary guide model based on the primary guide inputs,
wherein generating the first interaction plan is further based on the primary guide model.

20. A non-transitory computer-readable storage medium that stores instructions for an interaction engine of an Extended Reality (XR) system for guiding a therapeutic psychedelic experience that, when executed by a processor, cause the interaction engine to:
receive inputs from a participant of the therapeutic psychedelic experience;
determine a participant-XR system interaction model associated with engagement between the participant and the XR system based on participant inputs;
determine a first therapeutic goal associated with the participant based on participant inputs; and
generate a first interaction model comprising a first conversation component and an augmented or immersive element component based on the participant-XR system interaction model and the first therapeutic goal.
